(19) United States
(12) Patent Application Publication
(10) Pub. No.: US 2007/0269401 A1
(43) Pub. Date: Nov. 22, 2007

(54) Device for Distributing and/or Controlling the Discharge of Unitary Products, Fitted Onto a Container, and For the In-Situ Treatment of its Internal Atmosphere

(75) Inventor: Benoit Portier, Bourges (FR)

Correspondence Address: Womble Carlyle Sandridge & Rice, PLLC;Attn: Patent Docketing 32nd Floor, P.O. Box 7037, Atlanta, GA, 30357-0037, US

(73) Assignee: AIRSEC S.A.S., Choisey Le Roi (FR)

(21) Appl. No.: 11/751,226

(22) Filed: May 21, 2007

Related U.S. Application Data
(60) Provisional application No. 60/816,645, filed on Jun. 27, 2006.

(30) Foreign Application Priority Data
May 19, 2006 (FR) 06/04539
May 16, 2007 (FR) PCT/FR07/00831

(51) Int. Cl. A61L 9/01

(52) U.S. Cl. 424/76.1

(57) ABSTRACT

The present invention relates to a device for distribution and/or controlling the discharge of unitary products, and for simultaneous in-situ treatment of the atmosphere of a container onto which it is fitted. The device comprises a lower surface provided with an entry opening, an upper surface provided with at least one main distribution opening, a guiding surface for the products to be distributed, and a means of securing the device. The device is made from a thermoplastic material comprising at least one thermoplastic polymer and at least one agent for treating gaseous pollutants present in the atmosphere of the container, by reaction with and/or adsorption of at least one existing gaseous pollutant and/or desorption of said agent in the atmosphere of the container.

### CROSS REFERENCE TO RELATED APPLICATIONS

This application claims priority to French Application No. 06/04539, filed on May 19, 2006, U.S. Ser. No. 60/816,645, filed Jun. 27, 2006, and to PCT Application No. PCT/FR2007/000831, filed May 16, 2007, the contents of which are incorporated by reference in their entireties.

### BACKGROUND OF THE INVENTION

1. Field of the Invention

The present invention relates generally to a device for distributing and/or controlling the discharge of unitary products in a solid state as well as to the in-situ treatment of the atmosphere of the container onto which it is fitted. The unitary products are typically stored loosely so as to be distributed preferably unit by unit and the assembly generally comprises the container and an integrated device that is closed by a sealing means at the open end of the container.

Unitary products of this kind can be, for example, pharmaceutical, cosmetic, nutritional, veterinary or diagnostic products, with the characteristic of having one greater dimension which is either the diameter or the length. In the case of pharmaceutical products these can be tablets, dragees, pills, lozenges, pastilles, granules or capsules.

Products of this kind can be sensitive to the gaseous pollutants present in the internal atmosphere of the container, such as for example humidity, oxygen and other pollutants.

Thus a device of this kind for the distribution and/or control of discharge and for the in-situ treatment of the gaseous atmosphere of a container, makes it possible on the one hand to avoid the undesired release of several unitary products at once during its use, and on the other hand to ensure the preservation of a suitable atmosphere in the storage container, including during the time of use, by means of the adsorbent, reactive or desorbent properties of the material used to make a device of this kind.

2. Description of Related Art

Numerous devices for distributing and/or controlling discharge at a specific opening, with regard to the shape of the distribution opening and fitting onto containers, are described in the technical literature, in particular in the form of patent applications and/or published patents.

Thus for example, document WO 2004/080366 describes a device for the distribution of products with an oblong shape having a main opening and at least one other secondary opening with an elongated shape and a corresponding container.

In this device, the selected object is taken and passes partially through the main distribution opening and remains slightly wedged there, so that it cannot fall out of the container, but is still accessible to the user from the side of the external surface of the device.

A container of this kind is provided with a removable sealing stopper, mounted by an interlocking or threaded connection.

The level of humidity in the internal atmosphere of the container is controlled by incorporating desiccating elements, which can be in the form of inserts or layers of materials molded onto the internal surface of the container and/or desiccating elements inserted into the stopper closing the container or positioned at the bottom of the container.

A different document (EP-756567) describes a container designed for medicines such as tablets provided with a distribution system.

A container of this kind for circular, flattened tablets comprises a storage compartment able to hold a specific volume of tablets. The storage compartment is in communication with the exterior of the container by way of a distribution device comprising one or more distribution openings. The distribution device which fits onto the neck of the container, comprises a storage chamber for a generally pulverulent dehydrating agent, which thereby protects the tablets inside the container from ambient humidity. A membrane which is porous to humidity holds the pulverulent dehydrating agent and at the same time enables the humidity contained in the tablets stored in the body of the container and/or the internal humidity of the container to be adsorbed by the pulverulent dehydrating material.

A storage chamber of this kind which receives a specific quantity of pulverulent dehydrating material can be formed around part of the distribution device in the form of a narrowing surface leading to the distribution passage. The other part of this storage chamber is comprised by the walls of the container or by the external walls of the distribution device.

Clearly, the flaws in the technical solution according to this closest prior art relate to the fact that there are three components, namely a distributing cone and external walls of the attachment, pulverulent dehydrating material and porous membrane.

The conditions for producing an assembly of this kind are complex and difficult to automatize for the kind of mass production required by this type of packaging. Moreover, a device of this kind has the disadvantage of having a modest exchange surface, due to its particular shape, with the dehydrating material being stored between two impervious walls, and a membrane forming the third wall, but the membrane is not turned towards the interior of the container.

A further document (U.S. patent application Ser. No. 11/048,268, Published U.S> Patent Application 2006/0169603) describes a container equipped with a reclosable cover made in two parts. One is a fixed part in one piece with the body of the container, having a distribution opening with a diameter which is smaller than the diameter of the body of the container on which the reclosable cover is fixed, and the other part is a pivoting mobile section, in one piece with the fixed part and articulated around the fixed part by means of a hinge, thus constituting the reclosable covering cap.

The assembly comprises, in its fixed part, internal housing in which an insert is fitted made from a dehydrating composition. The dehydrating insert with a circular shape comprises a plurality of ribs separated by blind holes providing a number of exchange surfaces and is able to collect the internal humidity in the container.

An assembly of this kind with a dehydrating function has a main disadvantage of being made in several parts, and thus requires complex and expensive assembly operations. It is also not suitable as the distribution device is elaborate and no longer makes it possible to reserve such a housing to hold the dehydrating insert.

It can be observed that none of the distribution devices of the prior art for fitting onto containers produce satisfactory results, not with regard to the distribution of the products for which they are designed, but with regard to the integration of other functions, such as the ability to regulate the internal atmosphere of a container, for example its humidity levels.

### SUMMARY OF THE INVENTION

A problem posed, which has not been solved by the prior art, is to produce a device for distribution and/or controlling discharge that is designed for containers storing solid loosely packed unitary products, that are sensitive to humidity. For example, once a device is provided not only with the ability preferably to control discharge and distribute products preferably unit by unit, but also is provided with means for the in-situ treatment of the internal atmosphere of the container. Such an in-situ treatment can be for example dehydration, the elimination of oxygen and/or other gaseous pollutants present in the atmosphere.

Thus, the present invention relates to a device for distributing and/or controlling the discharge of unitary products and for treating in-situ the atmosphere of a closed container by a sealing means. The sealing means is fitted onto the closed container and the unitary products are stored loosely therein. The device enables the passage of the products to be distributed from the interior to the exterior of the container. In one embodiment the device comprises:
a lower surface equipped with an entry opening;
an upper surface provided with at least one main distribution opening;
between the lower surface and the upper surface, a surface for guiding products to be distributed;
a means for fixing the device onto the container in which it is fitted;
   Wherein the device is made from a thermoplastic material comprising at least one thermoplastic polymer and at least one agent for treating at least one gaseous pollutant present in the gaseous atmosphere of the container by reaction with and/or adsorption of at least one gaseous pollutant present in the gaseous atmosphere and/or desorption of the agent in the gaseous atmosphere of the container.

The present invention also relates to a form of packaging comprising: the distribution device, a container fitted therewith, and a closing means for closing the container equipped with the device in a sealing manner.

In addition, the present invention relates to the use of a device for distribution and/or controlling the discharge of unitary products and for the in-situ treatment of the atmosphere of a container onto which it is fitted. The products are preferably stored loosely in the container and can be in particular, pharmaceutical, cosmetic, nutritional, veterinary or diagnostic in type, regardless of their physical shape, and they may or may not be sensitive to gaseous pollutants, for example humidity, and require a specific protected environment in relation to the exterior environment.

Additional objects, features and advantages of the invention will be set forth in the description which follows, and in part, will be obvious from the description, or may be learned by practice of the invention. The objects, features and advantages of the invention may be realized and obtained by means of the instrumentalities and combinations particularly pointed out in the appended claims.

### BRIEF DESCRIPTION OF THE DRAWINGS

The invention is explained in more detail in the following description of the figures with reference numbers, where the figures illustrate a particular dehydrating container according to the invention in an illustrative and non-restrictive manner.

FIG. 1 show a cross section of a device for distribution and/or controlling discharge
and for the in-situ treatment of the internal atmosphere of a container with a solid structure, according to the invention, fitted onto its container;

FIG. 2 shows a cross section of the device with a thin walled structure according to the invention, fitted onto its container;

FIG. 3 shows a cross section of a device according to the invention, with a thin walled structure, with a single off-centre main distribution opening;

FIG. 4 shows a view from above of the same device;

FIG. 5 shows a perspective view of a device for distribution and for the in-situ treatment of the internal atmosphere of a container for oblong objects;

FIG. 6 is a perspective view of a device where the guiding surface is hemispherical and comprises a central main distribution opening, and secondary openings opening into the main distribution opening;

The accompanying drawings, which are incorporated in and constitute a part of the specification, illustrate particular embodiments of the invention, and, together with the general description given above and the detailed description of certain embodiments given below, serve to explain the principles of the invention.

### DETAILED DESCRIPTION OF A PREFERRED EMBODIMENT

Thus, according to the present invention, there is provided a device for distribution and/or controlling the discharge and for the in-situ treatment of the atmosphere of the container in which it is fitted, permits at the same time:
the distribution, preferably unit by unit, of loosely packed products to be distributed;
the in-situ treatment of pollution in the internal atmosphere of the container, which may be polluted for example, by water vapor, oxygen (O₂), gaseous pollutants of an acidic nature such as sulphur dioxide (SO₂), hydrosulphuric acid (H₂S), carbon dioxide (CO₂), carbon monoxide (CO), nitrogen dioxide (NO₂), halogens, in particular fluorine, gaseous pollutants of an alkaline nature such as ammonia (NH₃), organic gaseous pollutants that generate or do not generate odours such as alcohols, aldehydes, ketones, mercaptans, alkenes, in particular ethylene, alkynes, alkanes, in particular methane (CH₄),
as well as the in-situ treatment of the internal atmosphere with a view to obtaining pleasing olfactory properties.

There can be, however, instances of using the packaged products when the latter need to be taken not unit by unit but in a small number. It is then possible to prioritize the discharge of a limited number of products instead of distribution unit by unit, i.e. accept the discharge of several products and ensure that the surplus can be reincorporated into the container by a suitable means.

A device for distribution and/or controlling discharge and for the in-situ treatment of the atmosphere according to the invention can be designed to have a solid structure, such as a stopper, or to have a structure with thin walls, which can both be achieved, for example, by using plastics technology.

A device for distribution and/or controlling discharge and for the in-situ treatment of the atmosphere advantageously comprises a lower surface, an upper surface and a peripheral surface. In the case of a structure with thin walls, the external peripheral surface delimits a peripheral skirt on which the securing means can be arranged if desired.

The device for distribution and/or controlling discharge and for the in-situ treatment of the atmosphere also preferably comprises an entry opening, located on the lower surface, and advantageously turned towards the interior of the container. There is also preferably included a guiding surface, with or without a line of symmetry, the surface of which can be extendable or not extendable, bearing against the entry opening, as well as an outlet opening which opens into or which forms the main distribution opening. The device also preferably has a means for being secured onto the container on which it is fitted.

Entry Opening:

According to the present invention a device for distribution and/or controlling discharge and for the in-situ treatment of the atmosphere preferably comprises an entry opening on its lower surface, upstream of the guiding surface, and oriented towards the interior of the container, which can have, for example, a circular, elliptic or polygonal profile following the profile of the container. The profile can also be equal to or smaller than the internal profile of the container or of the neck of the container when the container is equipped therewith.

Main Distribution Opening:

The device for distribution and/or controlling the discharge and for the in-situ treatment of the atmosphere, according to the present invention comprises a main distribution opening arranged on its upper surface. Said opening makes it possible to distribute the products preferably unit by unit.

A main distribution opening can have, for example, a circular, elliptic or polygonal profile, or can have a completely different shape according to the shape and the dimensions of the products to be distributed. The opening generally has dimensions which enable the passage preferably of only one object at a time. Thus, if the distribution opening is circular, its diameter is generally close to the size or diameter of the product to be distributed.

A main distribution opening can be arranged in the plane or set back from the plane of the upper surface of the device or be placed in any desired arrangement.

Moreover, a main distribution opening can be positioned in a possibly preferential manner in relation to the axis of the container for example, in the center, that is in the line of axis of the device for distribution and/or controlling discharge. In other configurations, the distribution opening can be positioned outside of the line of symmetry of the device, such as, for example, at the periphery of its upper surface.

In more elaborate configurations, for example, for products with an oblong shape, a main distribution opening can possibly be connected with other secondary distribution openings, such as a type with lips or a different type, opening into the main distribution opening.

Guiding Surface:

The device for distribution and/or controlling the discharge and for the in-situ treatment of the atmosphere according to the present invention preferably comprises a guiding surface situated between a lower surface equipped with an entry opening and an upper surface equipped with at least one main distribution opening. The guiding surface typically opens directly into the main distribution opening or by way of a conduit. Regardless of the type of structure selected, either solid or with thin walls, the guiding surface is preferably in the form of a wall delimiting a hollow space with a spherical surface, such as a dome, or a spherical, truncated, truncated-cylindrical, truncated parabolic, truncated hyperbolic zone. The guiding surface can be, for example, convex or concave, with straight line or oblique revolution, i.e. generated by a rectilinear line or a curved line if desired.

The hollow space defined in this way has a height and base, whereby the relationship between the height and the dimension of the base can be very low, or even zero in the extreme instance of a plane guiding surface.

The guiding surface forming the wall is open at its two ends. One of the openings, generally the largest, is oriented towards the interior of the container and is the entry point of the device for the products to be distributed. The other opening opens into or constitutes the distribution opening or discharge control of said products, by nature of its geometry.

In one particular, extreme configuration the guiding surface can be a plane surface comprising the main distribution opening.

For example, in the case of a guiding surface with a conical shape, the angle at the summit of the cone can be between 30 and 180°, and preferably between 45 and 135°.

According to the mode the distribution device is functioning, when the distribution of the packaged products is required, the user inverts the direction of the distribution device by turning it upside down, so that the stock of products to be distributed is positioned above said device and is in contact with the guiding surface.

By inverting the device and thereby by the action of gravity, the products to be distributed come successively into contact with the guiding surface. Then with the distribution opening with adjusted dimensions, the passage preferably permits one object at a time to be distributed.

Means for Securing the Device onto the Neck of the Container:

A device for distribution and/or controlling discharge and for the in-situ treatment of the gaseous atmosphere according to the present invention also preferably comprises a means for securing the device onto the container on which it is fitted.

Preferably, the securing means is positioned on the external peripheral surface of the device in the case of solid structure, or on the peripheral skirt in the case of a structure with thin walls.

Various different securing means can be considered, such as for example, a force fit, a clipping connection with notch and groove on one or other of the existing surfaces, a differential thermal shrinkage, a screw connection, catching connection, etc. However, a preferred securing means is a force fit between the preferably cylindrical external surface of the device and the preferably cylindrical internal surface of the neck of the container.

In order to facilitate the fixing of the discharge control device onto the neck of the container by means of a force fit or clipping connection (which are modes of connection which are entirely based on elastic deformation of the device and which result from the mechanical properties of the thermoplastic material in which it is made), a slight modification of the shape of the device can be made. This modification can include providing at least one partial, radial slit in an upper peripheral surface of the device.

This partial slit will close partially when the discharge control device is introduced into the neck of the container, and will return to its initial position once the device has been positioned into the container's neck, at the level of the notch and groove.

The inclusion of at least one partial slit increases the capacity of reversible elastic deformation of the discharge control device during its introduction into the container's neck. Two rims are generally present in at least one partial slit, and capable of coming together during the introduction of the discharge control device into the container's neck.

The inclusion of the securing means makes it possible to provide a seal between the interior and exterior of the container when the external peripheral surface of the device forms an olive type bulge.

The securing means also preferably comprises specific means which make it possible to position the device vertically in relation to the neck of the associated container, by preventing vertical movement, once the device is put in place, in particular in the case of a force fit.

The specific means employed can be formed, for example, by a flange, bearing against the neck of the container a circular housing provided in the neck of the container, or by any other desired means.

The upper surface of the device after its introduction into the neck of the container, is at most flush with the annular surface of the neck or bears against this surface via a support means which may be a flange, whose diameter is greater than the internal diameter of the neck of the container and is at most equal to the external diameter of the neck of the container. The support means can be positioned at the level of the upper surface of the device and prevents the device being driven too deeply into the neck of the container and ensures precise vertical positioning.

Specific means of this kind can also include a reverse-lock stop positioned at the level of the lower surface of the device preventing the device from coming out of the neck of the container onto which it is fixed.

The lower stop can be in the form of a collar or segments of a collar providing a certain degree of elasticity to be introduced, by deformation, to the interior of the neck of the container. The stop can then relax and act like a non-return flap by coming to bear on the base of the neck of the container. This kind of elasticity and recovery of shape are ensured by the resilient material chosen to make the device and by the structure of this reverse-lock stop.

Several connecting means comprising the securing elements and positioning elements of the device relating to the neck of the container can be used together at the same time.

Thermoplastic Material Used for the Production of the Device:

According to the present invention, a device for distribution and/or controlling discharge and for the in-situ treatment of the internal atmosphere of a container is preferably made by injection techniques using thermoplastic materials comprising at least one thermoplastic polymer and at least one agent for treating the gaseous atmosphere by reaction with, and/or adsorption of at least one gaseous pollutant, and/or desorption of said agent in the internal atmosphere of the container.

A thermoplastic material formulated in this way acquires porosity due to the presence of one or more treatment agents which gives them the ability to exchange with some of the gaseous constituents concerned, such as the pollutants present in the gaseous atmosphere of the container, or to liberate the treatment agent into the atmosphere.

A suitable method of producing the device can also, in more simple structures, be single or multilayer extrusion, followed by thermoforming, then punching and cutting to obtain the said device. Injection-blow molding can also be considered. However, injection molding is preferable.

Thermoplastic Polymers Introduced into the Thermoplastic Materials According to the Invention:

A thermoplastic polymer as used to produce a device for distribution and/or controlling discharge and for the in-situ treatment of the internal atmosphere of a container, according to the present invention, are preferably selected from a group comprising high and low density, radical or linear polyethylenes, copolymers of ethylene such as ethylene vinyl acetates, ethylene ethyl acrylates, ethylene butyl acrylates, ethylene maleic anhydrides, ethylene alpha olefines, regardless of the methods of polymerisation or modification by grafting, homo polypropylene and copolymers, polybutene-1, polyisobutylene.

One or more polyolefines are preferably chosen for cost reasons and because they are easy to use.

However, other polymers can also be considered such as, polyvinyl chloride, copolymers of vinyl chloride, polyvinylidene chlorides, polystyrenes, copolymers of styrene, derivatives of cellulose, polyamides, polycarbonates, polyoxymethylenes, polyethylene terephthalates, polybutylene terephthalates, copolyesters, polyphenylene oxides, polymethyl methacrylates, copolymers of acrylate, fluoride polymers, polyphenylene sulphides, polyarylsulphones, polyaryletherketones, polyetherimides, polyimides, thermoplastic elastomers, polyurethanes, phenol resins, melamine resins, urea resins, epoxy resins and unsaturated polyester resins.

Biodegradable thermoplastic polymers can also be used such as for example polylactic acids (PLA).

Combinations of these polymers can also be produced in a mixture with polymers of polar groups, such as polyvinyl alcohols. Foaming agents can be used to expand the thermoplastic material and create open or closed cells.

Treatments Agents Used in the Thermoplastic Materials According to the Invention:

A treatment agent used by introduction into and mixture with the thermoplastic polymer to produce a device for distribution and/or controlling discharge and for the in-situ treatment of the internal atmosphere of a container according to the invention, can advantageously be selected from known reaction agents and/or gaseous pollution adsorbing agents such as dehydrating agents, oxygen collectors, odour collectors, collectors of ammonia (NH₃), alcohols, aldehydes, ketones, sulphur dioxide (SO₂), hydrosulphuric acid (H₂S), mercaptans, alkenes in particular ethylene, alkynes, carbon dioxide (CO₂), carbon monoxide (CO), nitrogen dioxide (NO₂), alkanes, in particular methane (CH₄), halogens in particular fluorine, and/or desorption agents in the internal atmosphere of the container such as volatile olfactory agents.

Various different agents for the in-situ treatment of the internal gaseous atmosphere of a container equipped with a device according to the present invention can be cited by way of illustration:
A suitable dehydrating agent is an individual agent or a mixture of several dehydrating agents, selected from a group comprising silica gels, dehydrating clays, activated aluminas, calcium oxide, barium oxide, natural or synthetic zeolites, molecular or similar sieves, or deliquescent salts such as magnesium sulphate, calcium chloride, aluminium chloride, lithium chloride, calcium bromide, zinc chloride or the like. Preferably the dehydrating agent is a molecular sieve and/or a silica gel.
A suitable oxygen collecting agent is selected from a group comprising metal powders having a reducing capacity, in particular iron, zinc, tin powders, metal oxides still having the ability to oxidise, in particular ferrous oxide, as well as compounds of iron such as carbides, carbonyls, hydroxides, used alone or in the presence of an activator such as hydroxides, carbonates, sulphites, thiosulphates, phosphates, organic acid salts, or halogen salts of alkaline metals or alkaline earth metals, activated carbon, activated aluminas or activated clays.
Other agents for collecting oxygen can also be chosen from specific reactive polymers such as those described for example in the patents U.S. Pat. No. 5,736,616, WO9948963, WO98511758, all of which are incorporated herein by reference in their entireties, can be mixed with a thermoplastic polymer used to produce the device according to the present invention.
A suitable agent for collecting odors issuing from the organic gaseous pollutants is selected from a group comprising pulverulent activated carbon, hydrophobic zeolites, activated aluminas and activated clays, specific polymers such as copolymers of styrene and divinylbenzene, mixed with a thermoplastic polymer used to produce the device according to the present invention.
A suitable desorption agent liberated into the internal atmosphere of the container is a volatile olfactory agent such as an aromatic, perfume or scent, and is selected from a group of extracts from citrus fruits, in particular lemon, orange, grapefruit, mandarin, extracts of vanilla, essential oils, herbal extracts, spices and others. The preferred volatile olfactory components are highly volatile ingredients with a low boiling point.

The amount of treatment agent introduced into the thermoplastic polymer to produce a device according to the present invention expressed in percentage by weight can advantageously vary from 5% to 55% of the thermoplastic material used to produce the device, when the treatment agent is a reaction and/or adsorption agent.

The amount of treatment agent introduced into the thermoplastic polymer to produce a device according to the present invention, when these agents are desorption agents, in the internal atmosphere of the container can generally vary from 0.001% to 5% by weight, and preferably from 0.001 to 1% of the thermoplastic material used to produce the device.

The manufacture of the thermoplastic material used within the scope of the invention is preferably achieved by adding master batches comprising (in a concentrated proportion) the treatment agent or agents previously dispersed in a thermoplastic polymer matrix. The master batch is preferably in the form of granules which are them mixed with the virgin thermoplastic polymer granules in such a way that the quantities of treatment agent present in the polymer material match the amounts in percent by weight mentioned above.

Introduction by measurement of the master batches to be dispersed in the thermoplastic polymer can be performed directly at the injection machine, if the plasticization system is adequate to permit good homogenisation, or to be carried out in an extruder in order to obtain a material ready to use.

Master batches can be obtained by compound extrusion if desired.

The pulverulent mineral materials constituting the reaction agents or the adsorption agents are mixed dry with the virgin thermoplastic polymer granules, then extruded and granulated, generally in twin-screw extruders, with cutting at the top, according to methods that are well known in the art.

With regard to the desorption agents the volatile organic compounds (that is the extracts of aromas, perfumes and scents,) they can often be encapsulated in microcapsules, then dispersed in a thermoplastic matrix, and can also be in the form of a master batch to dilute in the thermoplastic polymer used to make the device. These kinds of master batches contain up to 20% of the olfactory compound in a generally polyolefinic matrix.

According to a different method of incorporation, the volatile olfactory compounds can be previously placed on an adsorbent mineral support, which is connected to a thermoplastic matrix for the production of a master batch.

Finally, other methods of production are also possible, as described for example in U.S. Pat. No. 6,334,974 (incorporated by reference) where the impregnation of a thermoplastic powder precedes the production of the master batch, the latter being added to the thermoplastic polymer to be transformed by injection into a device according to the present invention.

The stabling additives, colorants, sliding agents, demolding agents, adhesive agents or reinforced attachment agents can be added to the thermoplastic polymers used to make the device for distribution and/or control discharge and for the in-situ treatment of the atmosphere according to the requirements of the production.

The device for distribution and/or controlling discharge and for the in-situ treatment of the internal atmosphere can also be produced with a dual material structure, on all or part of the surface, so as, for example, to encourage or prevent the sliding of the unitary products to be distributed via the distribution opening or openings.

The device for distribution and/or controlling the discharge and for the in-situ treatment of the internal atmosphere can be a separate object to be inserted into the container, in a preferred configuration, and/or can be co-injected with the body of the container by means of dual material injection techniques well known from the art.

In cases where the device for distribution and/or controlling discharge and for the in-situ treatment of the internal atmosphere is co-injected with the body of the container, the device constitutes all or a part of the body of the container. The body of the container is then stopped by a sealing base. In this configuration the part extending the device to form a part of the body or container is made from an impermeable thermoplastic material. The part of the device forming in particular the guiding surface for the products to be distributed can be made from a thermoplastic material made from at least one thermoplastic polymer and from at least one agent for treating the gaseous atmosphere, having the capacity to exchange with some of the gaseous constituents considered to be pollutants that are present in the gaseous atmosphere of the container.

A container equipped with a device for distribution and/or controlling discharge and for the in-situ treatment of the internal atmosphere according to the present invention also preferably comprises a means for sealing the open end of the container, which can be a stopper and/or cover, designed to ensure a seal between the container and the stopper/cover. This seal can be obtained by a structure combining adjustment between male and female parts and adapted geometric shapes and possibly by utilizing the elasticity of the materials forming the container and the stopper and/or cover.

The sealing means can be connected to the body of the container by linking means. Said linking means can be a mechanical type hinge with a pin or an integral type, depending on whether the container is a two-piece container, i.e. it consists of a body and stopper/cover made separately and possibly made from two materials which can be different or identical in nature, or in one piece and therefore generally made from only one material.

The container equipped with its device for distribution and/or controlling discharge and for the in-situ treatment of the internal atmosphere according to the present invention can also comprise an inviolability control.

The container equipped with a device of the present invention can also mount a sealed protective cover on its neck so that the upper surface of the device is flush with or is slightly set back from the neck.

In a preferred use, the container can be first filled with the products to be distributed, and then a device according to the present invention can be incorporated, and optionally a protective seal can be applied. The container can then be closed by a sealing means such as a reclosable stopper type.

In a specific use, and when the device for distribution and/or controlling discharge and for the in-situ treatment of the internal atmosphere forms all or part of the body of the container, the body of the container can be filled with the products to be distributed and then sealed by a sealing base. The sealing means of the reclosable stopper and/or cover type in this configuration can thus be installed and closed prior to filling, and prior to the irreversible and definitive closure by the attached base.

Thus, a device of this kind for distribution and/or controlling discharge and for the in-situ treatment of the internal atmosphere, fitted onto a container, is used to distribute, preferably unit by unit, a stock of products to be distributed that are being stored prior to use and protected from the exterior atmosphere by treatment of the internal atmosphere of the container.

According to FIG. 1, the device (1) according to the invention comprises a main distribution opening (2) preceded by a guiding surface (3), with a line of symmetry, said surface (3) having an entry opening (4) which is the large base oriented towards the interior of the container (7), and an outlet opening (5) which opens into the main distribution opening (2), said device having a means for securing (6) onto the associated container (7).

The device is positioned vertically onto the neck of the container by means of a flange (8) and is retained to prevent it becoming detached by a shoulder (9) including collar segments.

The device is made from a thermoplastic material comprising:
42% by weight of a radical low density polyethylene produced by POLIMERI with the reference number Riblene MP 31;
8% by weight of an ethylene vinyl acetate produced by POLYMERI with the reference number Greenflex MQ40;
50% by weight of a molecular sieve produced by CECA with the reference number NK10AP designed to absorb humidity.

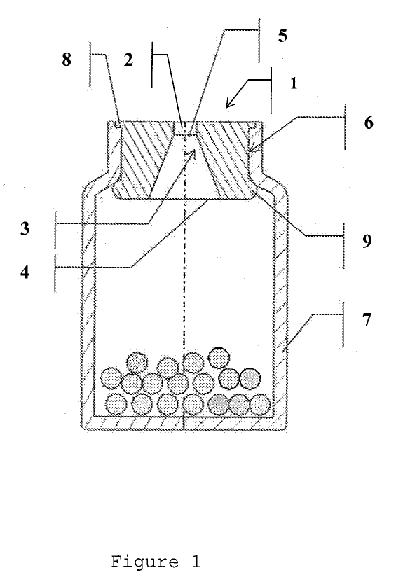

According to FIG. 2, the device (1) according to the invention comprises in the same manner a main distribution opening (2) preceded by a guiding surface (3), with a line of symmetry, said surface (3) having an entry opening (4) which is the large base oriented towards the interior of the container (7), and an outlet opening (5) which opens into the main distribution opening (2), the device having a securing means (6) which is a force fit, in the neck of the container (7). The device is positioned vertically on the neck of the container by means of a flange (8).

The device of figure is made form a thermoplastic material comprising:
74% by weight of a radical low density polyethylene produced by BOREALIS with the reference number FA6220,
25% by weight of a master batch available from Firmenich with the reference number N&A Orange 059427 A, for emitting a citrus orange perfume,
1% by weight of a master batch coloured orange available from Ciba with reference number PH5150

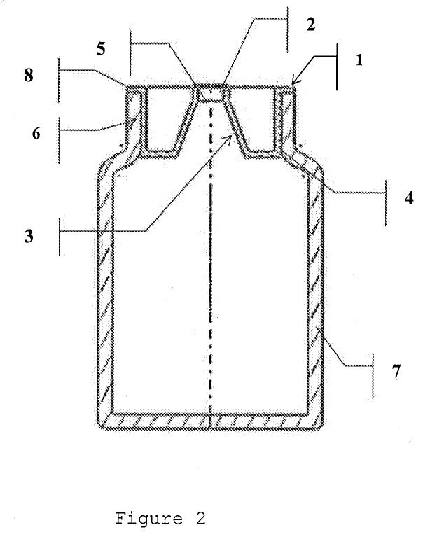

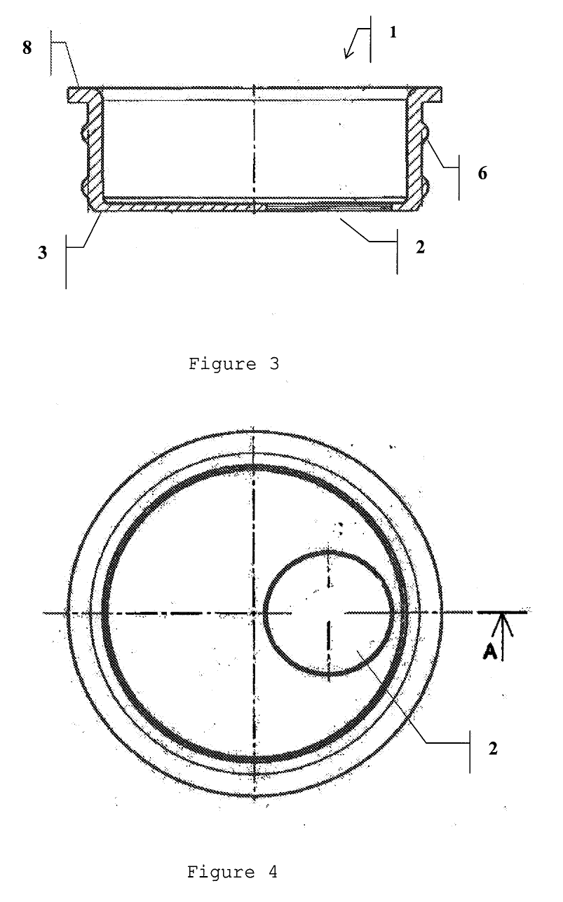

FIGS. 3 and 4 show a cross section of a device (1) according to the invention with a thin-walled structure, with a single off-centre main distribution opening (2), where the guiding surface (3) is plane. The securing means (6) is of the notch and groove type, providing a reverse-lock function. The flange (8) positions the device (1) vertically on the neck of the container (7).

The device is made from a thermoplastic material comprising:
42% by weight of a linear low density polyethylene;
8% by weight of a copolymer of ethylene vinyl acetate;
40% by weight of a molecular sieve produced by CECA with the reference number NK10AP designed to absorb humidity;
10% by weight of iron powder with oxidising ability, in the form of a master batch to absorb the existing oxygen.

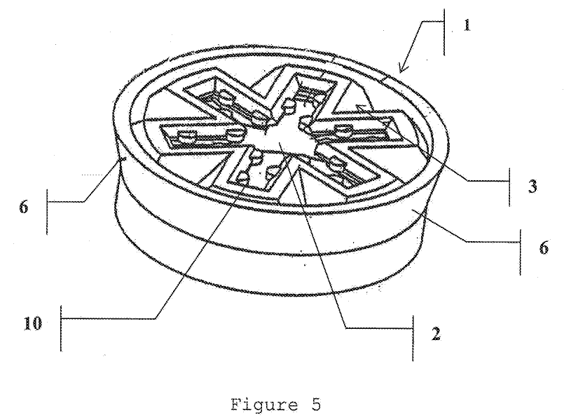

FIG. 5 shows a perspective view of a device for distribution and for the in-situ treatment of the internal atmosphere of a container, for oblong objects.

The device (1) with a thin-walled structure, comprises a main distribution opening (2), a series of elongated openings (10) dissecting each other and in a straight line, arranged radially relative to the line of symmetry of the device (1) and opening into the main distribution opening (2).

The guiding surface (3) has multiple planes connected two by two upstream of the secondary distribution openings (10) to form feeder openings which open into the main distribution opening (2) and into the secondary distribution openings.

The securing means (6) is of the force fit type which makes it possible to position the device (1) vertically on the neck of the container (7) by means of a truncated shape.

The device is made from a thermoplastic material comprising:
42% by weight of a radical low density polyethylene produced by POLIMERI with the reference number Riblene MP 31;
8% by weight of a copolymer of ethylene vinyl acetate produced by POLYMERI with the reference number Greenflex MQ40;
35% by weight of a molecular sieve produced by CECA with the reference number NK10AP designed to absorb humidity;
15% by weight of an activated carbon for absorbing odours.

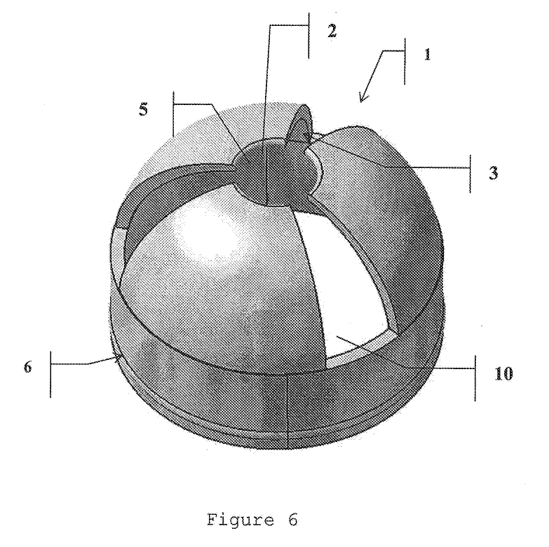

FIG. 6 shows a perspective view of a device (1), the guiding surface of which is hemispherical and comprises a central main distribution opening (2) and secondary openings (10) opening into the main distribution opening (2).

The device is made from a thermoplastic material comprising:
42% by weight of a radical low density polyethylene produced by POLIMERI with the reference number Riblene MP 31;
8% by weight of a copolymer of ethylene vinyl acetate produced by POLYMERI with the reference number Greenflex MQ40;
50% by weight of a molecular sieve produced by CECA with the reference number NK10AP designed to absorb humidity.

The devices and composition mentioned herein are interchangeable in terms of their specific embodiments and the compositions of the thermoplastic employed. Different components can be used in various other embodiments and using any desired thermoplastic material, including any of those described supra or others. This type of variation is contemplated by the present invention.

Additional advantages, features and modifications will readily occur to those skilled in the art. Therefore, the invention in its broader aspects is not limited to the specific details, and representative devices, shown and described herein. Accordingly, various modifications may be made without departing from the spirit or scope of the general inventive concept as defined by the appended claims and their equivalents.

All documents referred to herein are specifically incorporated herein by reference in their entireties.

The use of singular article terms including “an”, “a” and “the” can connote the singular or plural of the object that follows.

1. A device for distribution and/or controlling the discharge of unitary products and for the in-situ treatment of the atmosphere of a container closed by sealing means into which it is fitted, and in which said unitary products are stored loosely, said device permitting the passage of and said device products to be distributed from the interior to the exterior of the container, comprising:
a lower surface equipped with an entry opening;
an upper surface equipped with at least one distribution opening;
between the lower surface and the upper surface, a guiding surface for the products to be distributed;
a means for securing said device onto the container onto which it is fitted;
wherein said device comprises a thermoplastic material which comprises at least one thermoplastic polymer and at least one agent for treating at least one gaseous pollutant present in a gaseous atmosphere of the container by reaction with and/or adsorption of at least one gaseous pollutant present in said gaseous atmosphere and/or desorption of said agent in said gaseous atmosphere of the container.

2. A device according to claim 1, wherein the treatment agent present in the thermoplastic material is selected from agents processing a capacity for adsorbing water vapor.

3. A device according to claim 2, wherein the treatment agent is selected from the group consisting of silica gels, dehydrating clays, activated aluminas, calcium oxide, barium oxide, natural or synthetic zeolites, molecular sieves, and deliquescent salts.

4. A device according to claim 1, wherein the treatment agent present in the thermoplastic material is selected from agents having a capacity for reacting with and/or adsorbing oxygen.

5. A device according to claim 4, wherein the treatment agent is selected from the group consisting of metal powders, with a reducing property, metal oxides capable of oxidizing compounds of iron.

6. A device according to claim 1, wherein the treatment agent present in the thermoplastic material is selected from the group consisting of alkaline agents capable of reacting with and/or adsorb acidic gaseous pollutants.

7. A device according to claims 1, wherein the treatment agent present in the thermoplastic material is selected from the group consisting of acidic agents capable of reacting with and/or adsorb gaseous alkaline pollutants.

8. A device according claim 1, wherein the treatment agent present in the thermoplastic material is selected from the group consisting of agents capable of reacting with and/or adsorb organic gaseous pollutants.

9. A device according to claim 1, wherein the treatment agent present in the thermoplastic material is selected from the group consisting of agents capable of desorbing a volatile olfactory agent and said volatile olfactory agent is selected from the group consisting of extracts from citrus fruits.

10. A device according to claim 1, wherein the thermoplastic polymer used to produce the device is at lest one selected from the group consisting of polyethylenes, copolymers of ethylene, homo polypropylene, polybutene-1, polyisobutylene, polyvinyl chloride, copolymers of vinyl chloride, polyvinylidene chlorides, polystyrenes, copolymers of styrene, derivatives of cellulose, polyamides, polycarbonates, polyoxymethylenes, polyethylene terephthalates, polybutylene terephthalates, copolyesters, polyphenylene oxides, polymethyl methacrylates, copolymers of acrylate, fluoride polymers, polyphenylene sulphides, polyarylsulphones, polyaryl-etherketones, polyetherimides, polyimides, thermoplastic elastomers, polyurethanes, phenol resins, melamine resins, urea resins, epoxy resins and unsaturated polyester resins, biodegradable thermoplastic polymers, and polylactic acids (PLA).

11. A device according to claim 10, wherein the thermoplastic polymer used to produce the device comprises a polyolefine.

12. A device according to claim 1, wherein the amount of treatment agent introduced into the thermoplastic polymer is from 5% to 55% by weight of the thermoplastic material used, when the treatment agents comprise reaction agents and/or adsorption agents.

13. A device according to claim 1, wherein in the amount of treatment agent introduced into the thermoplastic polymer is from 0.001% to 5% by weight of the thermoplastic material used, when the treatment agents are capable of desorbing a volatile olfactory agent from an internal atmosphere of the container.

14. A device according to claim 1, wherein in the entry opening of the device has a circular, elliptic or polygonal profile.

15. A device according to claim 1, wherein in the entry opening of the device has a profile that is at most the same size as the internal profile of the neck of the container.

16. A device according to claim 1, wherein the guiding surface of the device is a spherical, truncated, truncated parabolic, truncated hyperbolic surface, the surface being concave, with straight line or oblique revolution.

17. A device according to claim 1, wherein the guiding surface is a plane surface comprising the main distribution opening.

18. A device according to claim 1, wherein the distribution opening has a circular, elliptic or polygonal profile.

19. A device according to claim 1, wherein the distribution opening is positioned in a plane of the upper surface of the device and at any point of said plane.

20. A device according to claim 1, wherein the distribution opening is set back from a plane of the upper surface of the device and at any point of said plane.

21. A device according to claim 19, wherein the distribution opening is in an axis line of the device.

22. A device according to claim 1, wherein the distribution opening is associated with at least one further secondary distribution opening that opens into said opening.

23. A device according to claim 1, wherein the securing means of the device is positioned on the peripheral external surface or skirt of said device.

24. A device according to claim 23, wherein the means for securing is at least one of a force fit, a clipping connection with notch and groove, a differential thermal shrinkage, a screw connection or a catching connection.

25. A device according to claim 24, wherein said securing means is a force fit connection or a clipping connection with notch and groove, and wherein said device includes at least one partial, radial slit in an upper peripheral surface thereof.

26. A device according to claim 1, further comprising a specific means permitting consistent vertical positioning in a flange abutting against a collar of the container or in a circular housing provided in a neck portion of the container.

27. A device according to claim 1, further comprising a specific reverse-lock means comprising a stop positioned at a level of the lower surface of said device.

28. A method for distributing products that are loosely packed, where the levels of humidity, oxygen and other pollutants need to be controlled by adding a volatile olfactory compound, said method comprising utilizing a device according to claim 1.

29. A method of claim 28, wherein said method involves unit by unit distribution.

30. A method of claim 28 further comprising inverting said device so that at least one product is in contact with said guiding surface and is thereby distributed.
